(19) United States
(12) Patent Application Publication
(10) Pub. No.: US 2004/0002724 A1
(43) Pub. Date: Jan. 1, 2004

(54) Navigable trocar with safety tip

(76) Inventor: Mark H. Falahee, Ann Arbor, MI (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/444,882

(22) Filed: May 23, 2003

Related U.S. Application Data
(60) Provisional application No. 60/382,838, filed on May 23, 2002.

(51) Int. Cl. A61B 17/34

(52) U.S. Cl. 606/185; 604/272

(57) ABSTRACT

A more navigable trocar includes an elongated curved shaft having a proximal end adapted for attachment to a drainage tube and a sharpened distal tip, and one or more recess pads closer to the proximal end, facilitating a more predictable grasping and manipulation to steer the trocar through a body. A pair of spaced-apart recess pads may be used, and they may be angled relative to one another to accommodate use of either hand by a surgeon. A different preferred embodiment includes a protective cap for a trocar comprising a polymeric or elastomeric material including a central portion, enabling the sharpened tip of the trocar to pierce and penetrate therethrough with the cap remaining outside the body. This embodiment may be used with or without the recess pad(s). The cap may further include a flattened distal end to stabilize and reduce movement as the tip of the trocar is pushed through the cap.

### REFERENCE TO RELATED APPLICATION

This application claims priority from U.S. Provisional Patent Application Serial No. 60/382,838, filed May 23, 2002, the entire content being incorporated herein by reference.

### FIELD OF THE INVENTION

This invention relates generally to surgical instruments and techniques and, in particular, to a trocar which is more easily manipulated and less prone to error.

### BACKGROUND OF THE INVENTION

Trocars are commonly used in surgical drainage tube placement. One example is the Allegiance Jackson-Pratt Hemovac System. Trocars are essentially long, smooth, large needles having an extremely sharp distal end and drainage tube connected to the proximal end. A protective sleeve generally covers the sharp tip, which is removed when the device is handed to the surgeon. It is inserted proximate to a wound/closure and then oriented through the body so as to emerge from the patient at a point where drainage is needed.

The difficulty with orienting trocars is that the devices are smooth and quite a bit of force is required to penetrate certain types of tissues. They are necessarily curved, such that, particularly when force is applied, it is difficult to predict where they will come out, requiring significant manipulation in some cases to ensure that the device is properly oriented. Even with a kocar or similar surgical clamp, it is difficult and hazardous to navigate the smooth shafts of existing trocars through soft tissue to the correct puncture sight at skin level. It is also hazardous to handle the trocars due to their sharp, unprotected tips.

### SUMMARY OF THE INVENTION

This invention improves upon the prior art by providing a more navigable trocar. One preferred embodiment includes an elongated curved shaft having a proximal end adapted for attachment to a drainage tube and a sharpened distal tip, and one or more recess pads closer to the proximal end, facilitating a more predictable grasping and manipulation to steer the trocar through a body. A pair of spaced-apart recess pads may be used, and they may be angled relative to one another to accommodate use of either hand by a surgeon.

A different preferred embodiment includes a protective cap for a trocar comprising a polymeric or elastomeric material including a central portion, enabling the sharpened tip of the trocar to pierce and penetrate therethrough with the cap remaining outside the body. This embodiment may be used with or without the recess pad(s). The cap may further include a flattened distal end to stabilize and reduce movement as the tip of the trocar is pushed through the cap.

### BRIEF DESCRIPTION OF THE DRAWINGS

FIG. 1 is a drawing of a preferred embodiment of this invention;

FIG. 2 is a drawing which shows an optional protective cap having a flattened protective cap;

FIG. 3A is a drawing which shows the embodiment of FIG. 2 in an initial position;

FIG. 3B is a drawing which shows the embodiment of FIG. 2, penetrating through tissue from within a surgical wound to the outside of a patient's body through the skin; and

FIG. 3C illustrates the way in which the flat protective top of the embodiment of FIG. 2 is pierced yet retained along the tubing and removed from the body.

### DETAILED DESCRIPTION OF THE INVENTION

This invention solves problems associated with prior art trocars in multiple ways. In one embodiment, a trocar according to the invention features navigational pads to allow a secure grip and steering capability using a kocar or similar surgical clamp. In a different embodiment, a silicone cap is provided which covers the trocar sharp tip but, in contrast to previous protective sleeves which are removed prior to insertion, making the device most hazardous at the point of surgeon utilization, the trocar according to this invention simply pierces through the protective cap, which includes a flared, flat top to facilitate piercing and penetration.

Reference is now made to the figures, wherein FIG. 1 shows a preferred embodiment of the invention generally at 100, which includes a curved shaft 110 having at least one pad to permit grasping with forceps or other tool or direct manual manipulation. In the preferred embodiment, two pads 112, 114 are provided and are offset at 60 degrees relative to one another, enabling the surgeon to grasp the shaft in either location without turning. Although two separate and distinct pads 112, 114 are shown, a single recessed area, perhaps including a square-, rectangular-, diamond- or trapezoid-cross-section. In any case, note that each pad is recessed below the outer surface of the shaft overall, so as not to interfere with movement of the shaft of through body tissues. Additionally, though optional, the bottom surface of each pad is preferably grooved or roughened to enhance gripping capability. Note further that the pad(s) are preferably located toward the proximal end of the device 100, where it attaches to tube 111, to maintain an optimal level of control while the majority of the trocar is navigated through the body. As a further alternative to one or more pads, the proximal end itself may have a hexagonal or other non-round cross-section to facilitate grasping, as indicated at 113.

The trocar 100 in FIG. 1 may include a conventional cover 120 to cover the sharpened tip 121. However, as an alternative, the cover 200 may alternatively be employed, with or without the recessed pads. That is, the cover 200 represents patentably distinct subject matter, and may be used with existing trocars.

In the preferred embodiment, the cover 200 includes a flattened distal portion 220 and, depending upon the material used, may include a thin section 222 between the flattened outer surface and the tip 230 of the trocar 210. These features allow the cap 200 to be placed against tissues inside the wound of the patient and the trocar 210 to be pushed through so that the tip is not exposed until it finally emerges from the body. This reduces hazards associated with exposure to the tip prior to entry and, if used in conjunction with the embodiment of FIG. 1, hazards are reduced as the trocar emerges from the body, since a more predictable navigation is possible.

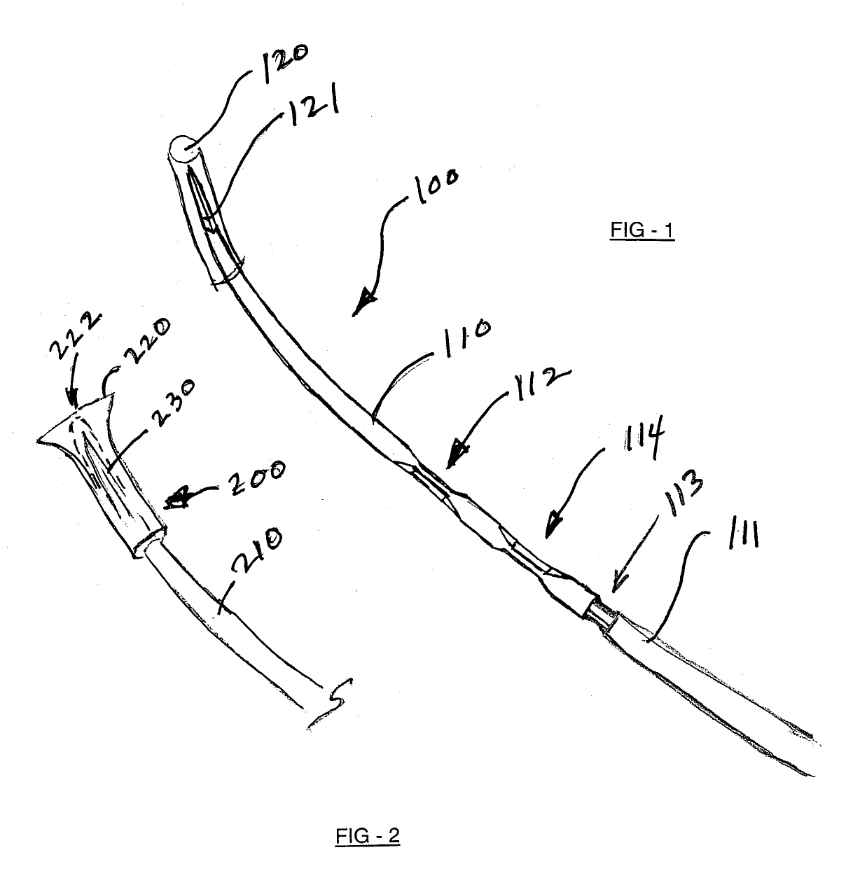

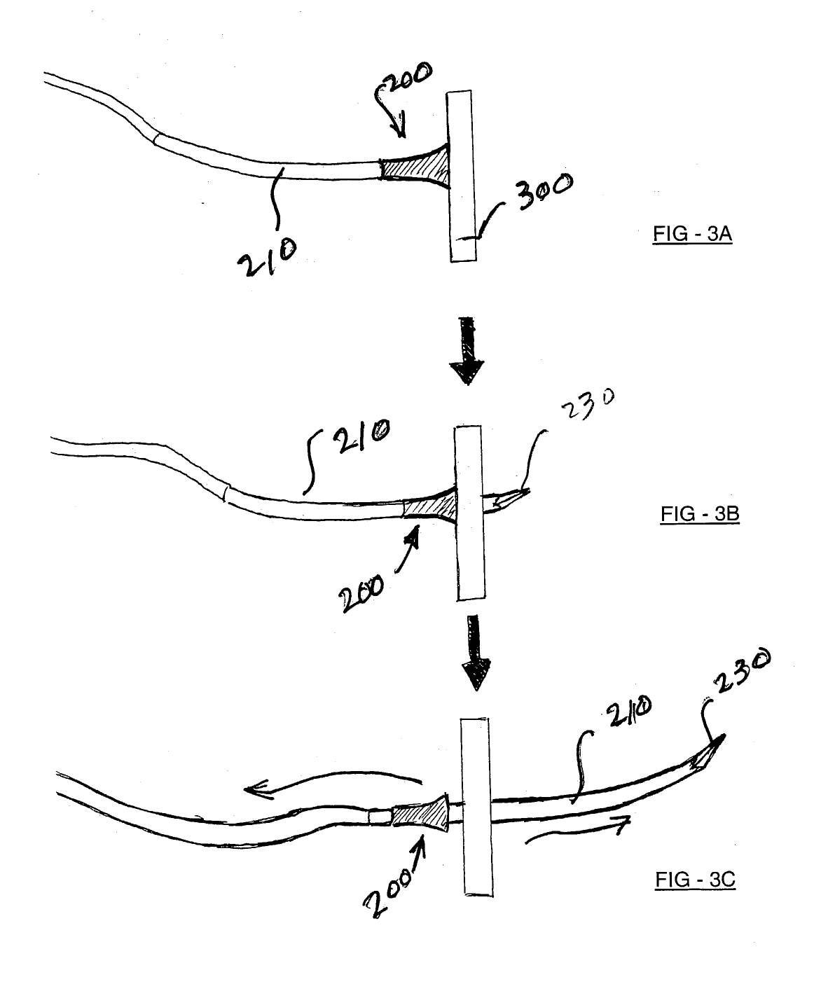

FIGS. 3A-3C show the way in which the protective cap 200 is used in a procedure through layers of tissue 300. In FIG. 3A, the flattened end of the cap 200 rests against tissues inside the wound of the patient, and the tip of the trocar shaft 210 has not yet penetrated therethrough. In FIG. 3B, the tip 230 has been pushed through the end 222 of the cap as shown in FIG. 2, and through the layers 300, now exiting the body. In FIG. 3C, the trocar continues to move through the body, with cap 200 sliding back onto the tube, enabling it to be removed at the proximal end thereof.

The materials used for the various embodiments of this invention may be conventional, in the sense that the trocar may be made of a metal alloy, and the protective cap made with silicone or other polymeric/elastomeric material. The grooved/roughened surfaces of the pads, particularly for use with manual manipulation, may include a diamond surface or even diamond-like coating, as appropriate. Additionally, the needle may be made with a hardened plastic, such as polycarbonic, or the like, with the distal tip being perhaps treated in some manner to enhanced hardening and/or sharpness.

I claim:

1. A more navigable trocar, comprising:
an elongated curved shaft having a proximal end adapted for attachment to a drainage tube and a sharpened distal tip; and
one or more non-round cross-sections closer to the proximal end, facilitating a more predictable grasping and manipulation to steer the trocar through a body.

2. The trocar of claim 1, wherein the non-round cross-sections include one or more spaced-apart recessed pads.

3. The trocar of claim 2, including a plurality of pads with surfaces which are angled relative to one another to accommodate use of either hand by a surgeon.

4. A protective cap for a trocar comprising a polymeric or elastomeric material including a central portion enabling the sharpened tip of the trocar to pierce and penetrate therethrough with the cap remaining outside the body.

5. The cap of claim 4, further including a flattened distal end to stabilize and reduce movement as the tip of the trocar is pushed through the cap.
